(19) United States
(12) Patent Application Publication
(10) Pub. No.: US 2014/0358241 A1
(43) Pub. Date: Dec. 4, 2014

(54) TOTAL KNEE PROSTHESIS, AND SET OF MODULAR ELEMENTS MAKING IT POSSIBLE TO OBTAIN SUCH A PROSTHESIS

(71) Applicant: Jacques AFRIAT, Narbonne (FR)

(72) Inventor: Jacques AFRIAT, Narbonne (FR)

(21) Appl. No.: 13/909,375

(22) Filed: Jun. 4, 2013

(51) Int. Cl. A61F 2/38

(52) U.S. Cl. CPC A61F 2/3877; A61F 2/3859
USPC 623/20.19

(57) ABSTRACT

The present invention concerns to a total knee prosthesis including a femoral implant having an anterior wall that forms the upper portion of a prosthetic trochlea and two curved branches extending from the lower edge of said anterior wall, delimiting the lower portion of the prosthetic trochlea between them and forming condylar surfaces between them. The trochlea has a middle furrow bordered by two rounded convex surfaces. The trochlea has in its upper portion a flexion of approximately 50°. The trochlea has a depth smaller than the height of the longitudinal middle rib comprised by the patella and has a width larger than that of that same rib, such that the patella comes into contact with the femoral implant essentially by that rib and said concave side faces are situated at a distance from said rounded convex surfaces. In the lower portion, the trochlea has a depth greater than the height of said rib and has a width substantially equal to that of said same rib, such that the patella comes into contact with the femoral implant essentially by said concave side faces, and said rib is at a distance from the bottom of the trochlea.

### TECHNICAL FIELD

The present invention relates to a total knee prosthesis, and a set of modular elements making it possible to obtain said prosthesis.

### BACKGROUND OF THE INVENTION

A total prosthesis of the knee traditionally comprises a femoral implant and a tibial implant made from metal, and an intermediate plate made from a material favoring sliding, in particular high-density polyethylene. Such a prosthesis also frequently comprises a patellar implant designed to be fastened on the bony patella after suitable resection thereof.

The femoral implant has a curved shape capable of surrounding the lower end of a femur, resected accordingly; it comprises an anterior wall forming the upper part of a prosthetic trochlea, and two curved branches extending from the lower edge of said anterior wall, these two branches defining, between them, the lower part of the prosthetic trochlea and forming condylar surfaces.

The tibial implant forms an upper wall for receiving said intermediate plate.

The patellar implant is frequently made up of a polyethylene piece anchored on the duly resected bony patella, resurfacing that bony patella.

It has been seen that with many existing prostheses, it is necessary to resurface the bony patella using a patellar implant. In fact, without such resurfacing, the prosthetic joint would have abnormal kinematics and stress peaks, and the patient would be exposed to pain and abnormal rapid wear of the bony patella would occur.

Another drawback of the existing prostheses is the generation of a fibrous nodule at the junction of the quadricipital tendon and the bony patella (phenomenon known as “clunk syndrome”).

### OBJECTS OF THE INVENTION

The present invention aims to resolve these essential drawbacks by providing a knee prosthesis in which a bony patella or a patellar implant can cooperate under optimal conditions with the prosthetic femoral implant, and in which the phenomenon of the generation of a fibrous nodule at the junction of the quadricipital tendon and the bony patella is avoided.

Another aim of the invention is to provide a set of modular elements making it possible, from the same basic elements, to obtain such a prosthesis, either non-posterior stabilized, i.e., implanted with preservation of the posterior cruciate ligament, or a posterior stabilized primary or initial prosthesis (i.e. for a first implantation), or a revision posterior stabilized prosthesis (i.e. for the replacement of a previously implanted prosthesis).

### SUMMARY OF THE INVENTION

The concerned prosthesis includes a femoral implant comprising an anterior wall that forms the upper portion of a prosthetic trochlea, and two curved branches extending from the lower edge of said anterior wall, delimiting the lower portion of the prosthetic trochlea between them and forming condylar surfaces between them;

the trochlea has, in transverse cross-section, a “gull-wing” shape, i.e., it has a middle furrow bordered by two rounded convex surfaces,

the trochlea has, between its anterior and posterior ends, the following evolving shape:

in its upper portion, i.e., its portion cooperating with the patella for flexion of the joint going from approximately 0° to approximately 50°, the trochlea has a depth smaller than the height of the longitudinal middle rib comprised by the patella with respect to the concave side faces bordering that rib and has a width larger than that of that same rib, such that the patella comes into contact with the femoral implant essentially by that rib and said concave side faces are situated at a distance from said rounded convex surfaces; said depth increases gradually toward the lower portion of the trochlea, until it corresponds to the height of said rib at the junction of said upper portion of the trochlea with said lower portion of that trochlea, and said width decreases gradually toward said lower portion of the trochlea, until it corresponds, at that same junction, to the width of said rib;

in its lower portion, i.e., the portion of the trochlea cooperating with the patella for flexion of the joint going from approximately 50° to approximately 120°, the trochlea has a depth greater than the height of said rib with respect to said concave side faces and has a width substantially equal to that of said same rib, such that the patella comes into contact with the femoral implant essentially by said concave side faces, said round convex surfaces, and said rib is at a distance from the bottom of the trochlea.

It will be understood that the term “patella” used above covers both the native bony patella, when the latter is preserved as such, and a resurfacing patellar implant, when such an implant is used.

Thus, in the prosthesis according to the invention, a progressive transfer occurs from the surface of the patella coming into contact with the femoral implant, said transfer being done from the central rib, which is essentially active at said upper portion of the trochlea, toward said concave side faces, which are active at said lower portion of the trochlea.

It results from the structure that the patella has, in said upper portion of the trochlea, a possibility of transverse translation and tilting with respect to the trochlea in the transverse plane, made possible by the space existing between said concave side faces and said rounded convex surfaces. This possibility of transverse translation and tilting is completely favorable to optimal cooperation of the patella with the femoral implant and therefore optimal operation of the prosthetic joint.

In said lower portion of the trochlea, this possibility of translation and tilting is eliminated by the arrival of said concave side faces bearing against said round convex surfaces, which is also completely favorable to optimal cooperation of the patella and the femoral implant. Furthermore, this arrival of said concave side faces bearing against said round convex surfaces makes it possible to keep the junction of the quadricipital tendon and the patella at a distance from the lower edge of said anterior portion of the femoral implant; as a result, the generation of a fibrous nodule at said junction area is greatly reduced, or even eliminated, and the rib of the patella is also at a distance from said lower edge and therefore does not risk undergoing a grating effect produced by said lower edge. In fact, said lower edge may be relatively aggressive and may have a significant impact on the generation of a fibrous nodule and on wear of the patella.

Preferably, the prosthesis comprises a patellar implant including a base designed to be fastened to the bony patella, duly resected, and a sliding element designed to be mounted on the base, with the possibility of pivoting with respect thereto; the sliding element forms, on the face thereof designed to come into contact with the femoral implant, a longitudinal rib and two concave side faces situated on either side of that rib, reproducing the rib and homologous faces of a bony patella, the rib having a rounded shape in the transverse plane, and its flanks being arched so as to connect continuously with said concave side faces; said rib and said concave side faces also have, in a sagittal plane, a concave curve substantially corresponding to the curve of the trochlea in that same sagittal plane.

Preferably, said anterior wall of the femoral implant forms an extended bottom of the trochlea, i.e., extends in the direction of the lower portion of the trochlea such that the lower edge of that interior wall is at the portion of the trochlea cooperating with the patella for flexion of the joint of approximately 60° to 65°.

In other words, the trochlea has a bottom extending beyond the aforementioned area by the 50° of flexion, from which said rib loses its contact with said bottom; as a result, when, during an extension movement of the joint, the lower edge of said interior wall reaches the level of said rib, said rib is at a distance from the edge that is sufficient to eliminate any risk of the grating effect; it is only then, around 50° of flexion, that said rib will again come into contact with the bottom of the trochlea, whereas the majority of it will then have been exceeded by said lower edge.

Preferably, the patellar implant comprises means for limiting the pivoting of the sliding element with respect to said base, preventing the pivoting of that sliding element beyond approximately 20° on either side of the neutral position in which said rib is parallel to the trochlea.

According to one preferred embodiment of these limiting means, said base comprises a slug protruding from its face receiving said sliding element, and said sliding element comprises a lumen in the shape of an arc circle centered on the pivot axis, capable of receiving the slug when the sliding element is mounted on said base.

The set of modular elements according to the invention comprises, to obtain a posterior stabilized primary or initial prosthesis:

a femoral implant including a posterior intercondylar bar and having said extended trochlea bottom, said anterior wall of said femoral implant having, on the underside of its anterior wall, an interlocking prominence;

a first intermediate plate including a posterior stabilization stud; and

a first removable intercondylar cage, capable of housing said posterior stabilization stud, whereof an anterior end has an interlocking recess capable of allowing interlocking of the interior end on said interlocking prominence.

The extension of said anterior wall of the femoral implant making it possible to form the extended trochlea bottom makes it possible to form said interlocking prominence on the anterior-inner face of said femoral implant, and said prominence makes it possible to position the intercondylar cage on the femoral implant easily and quickly. The intercondylar cage, thus fitted on said prominence, is completely immobilized with respect to the femoral implant and can be used as a solid base to assemble a medullary anchoring rod to the femur.

The set of modular elements also comprises, to obtain a posterior stabilized revision prosthesis, a second intercondylar cage, with a height greater than that of said first intercondylar cage, and a second intermediate plate comprising a posterior stabilization stud with a height greater than that of the posterior stabilization stud of said first intermediate plate, the anterior end of said second intercondylar cage having an interlocking recess capable of allowing the interlocking of said anterior end on said interlocking prominence.

One or the other of said first or second intercondylar cages may thus be placed on the femoral implant depending on whether it involves forming a primary or initial prosthesis or a revision prosthesis.

The upper wall of said first and/or said second intercondylar cage advantageously comprises means for mounting a rod or a medullary anchoring stud according to a plurality of possible positions in the anteroposterior direction.

The set of modular elements may also comprise, in particular to obtain a posterior stabilized revision prosthesis, at least one medullary anchoring rod and at least one sleeve with a flared or conical base portion, capable of being engaged on said rod.

The set of modular elements may also comprise at least one additional intermediate plate with no posterior stabilization stud, in particular two additional plates, one of which comprises deep dish surfaces.

### BRIEF DESCRIPTION OF THE FIGURES

The invention will be well understood, and other features and advantages thereof will appear, in reference to the appended diagrammatic drawing, showing, as non-limiting examples, several modular elements from the set of elements it concerns and two prostheses obtained using said elements.

FIG. 1 is a perspective view of several elements making it possible to obtain a posterior stabilized primary or initial prosthesis, including a patellar implant;

FIG. 2 is a view of said prosthesis, similar to FIG. 1, the prosthesis being in an extension position of the joint;

FIG. 3 is a view of the prosthesis in longitudinal cross-section, in an extreme flexion position;

FIG. 4 is a perspective view of the femoral implant;

FIG. 5 is a perspective view of that same femoral implant, from another angle;

FIG. 6 is an enlarged perspective view of a sliding element comprised by the patellar implant;

FIG. 7 is a top view of the prosthesis, in the extension position, with a cross-section passing through the axis of a pivot stud comprised by the patellar implant;

FIGS. 7A to 7D are partial views of the prosthesis similar to FIG. 7, respectively in a first position of transverse translation of the patellar implant with respect to this femoral implant, and a second position of said transverse translation, in a first tilting position of the patellar implant with respect to the femoral implant, and in a second position of said tilting;

FIGS. 8 to 11 are views of the prosthesis similar to FIG. 7, in flexion positions of the joint respectively at 30°, 60°, 90° and 120 °;

FIG. 12 is a perspective view of several elements making it possible to obtain a posterior stabilized revision prosthesis, including a patellar implant; and

FIG. 13 is a view of said revision prosthesis, similar to FIG. 12.

### DESCRIPTION OF THE PREFERRED EMBODIMENT

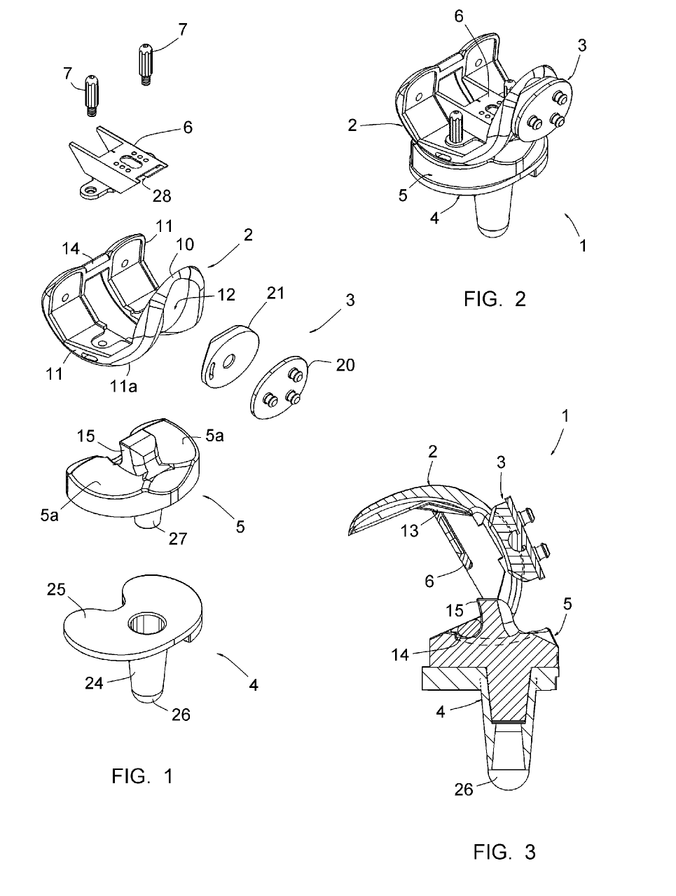

FIG. 1 shows a set of elements making it possible to obtain the posterior stabilized primary or initial prosthesis 1 shown in FIGS. 2 and 3.

This set of elements comprises a femoral implant 2, a patellar implant 3, a tibial implant 4, an intermediate sliding plate 5, an intercondylar cage 6 and two anchoring studs 7.

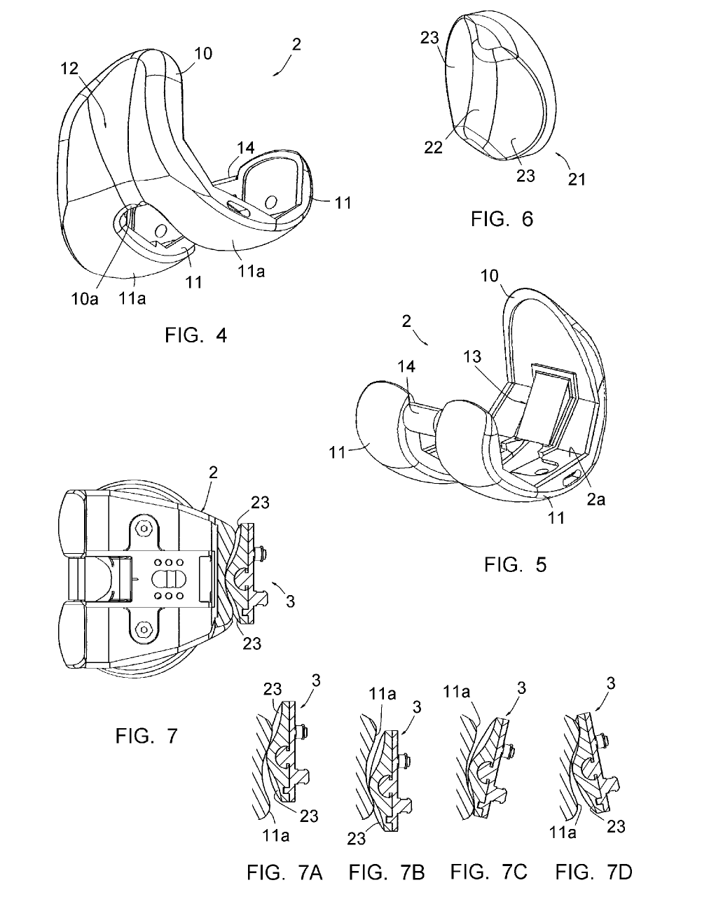

The femoral implant 2 is more particularly shown in FIGS. 4 and 5. It is formed by a single piece of metal having a curved shape capable of surrounding the lower end of a femur, resected accordingly. It comprises an anterior wall 10 and two curved branches 11 extending from the lower edge of said anterior wall 10.

On its outer side, the wall 10 forms the upper part of a prosthetic trochlea 12, the lower portion of which is delimited by the two branches 11 between them. The wall 10 thus has a lower edge 10a extending transversely to the branches 11; as more particularly shown in FIG. 4, and as will be understood in reference to FIGS. 9 and 10, the wall 10 extends relatively far toward the lower portion of the trochlea 12, such that its lower edge 10a is located at the portion of the trochlea 12 cooperating with the patellar implant 3 for flexion of the joint of approximately 60° to 65°.

On its inner side, the wall 10 has a middle interlocking prominence 13, formed by an inclined wall beginning at an inner distal face 2a of the femoral implant 2 and extending as far as the base of an upper inner face of said wall 10.

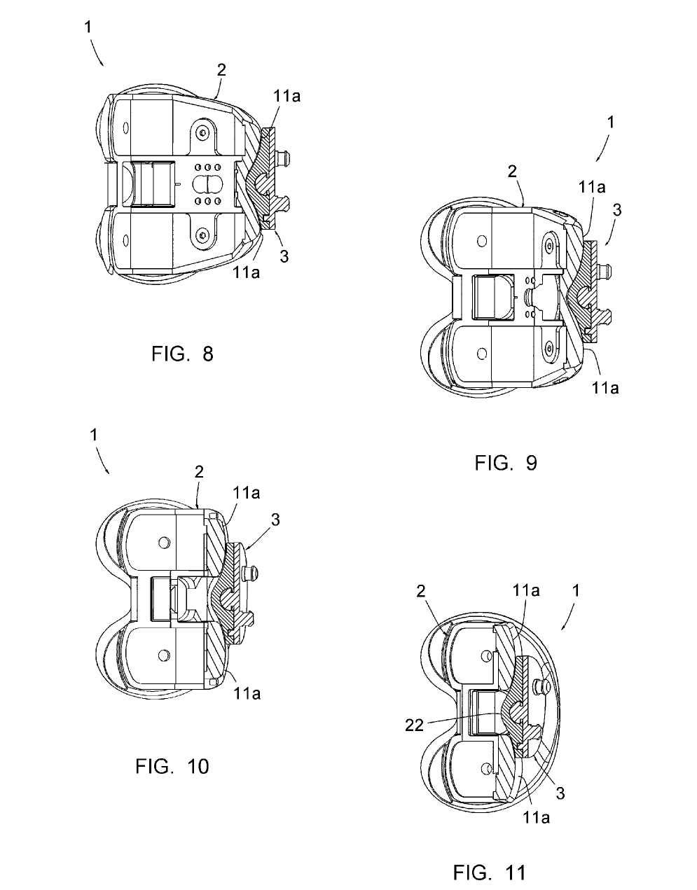

The curved branches 11 form condylar surfaces 11a having, in a direction transverse to the extension direction of those branches 11, a constant curve radius over the entire length of the branches 11 (see the cross-sections of the surfaces 11a shown in FIGS. 8 to 11).

The branches 11 are, at their posterior ends, connected to each other by a posterior stabilization bar 14 designed to come into contact with a posterior stabilization stud 15 comprised by the intermediate plate 5.

The patellar implant 3 is formed by assembling a base 20 designed to be fastened to the bony patella and a sliding element 21 designed to be mounted on that base, with the possibility of pivoting relative thereto.

The base 20 comprises, on the face thereof designed to receive the sliding element 21, a shouldered stud forming a pivot axis for that element 21 an off-centered slug. On its opposite face, it comprises three anchoring studs to the bony patella.

The sliding element 21 has a hole emerging in its face designed to be received by the base 20, which allows pivoting snapping of the element 21 on said shouldered stud of that base, and a lumen in the shape of an arc of circle centered on the axis of the hole, capable of receiving said slug of the base 20 when the sliding element 21 is thus mounted on said base 20 (see FIG. 7). Said slug and said lumen constitute means for limiting the pivoting of the sliding element 21 with respect to the base 20.

As shown more particularly in FIG. 5, the sliding element 21 forms, on the face thereof designed to cooperate with the femoral element 2, a longitudinal rib 22 and two concave side faces 23 situated on either side of said rib 22. The rib 22 has a rounded shape seen in cross-section in a transverse plane (see FIGS. 7 to 11), and its flanks are arched so as to connect continuously to the faces 23. The sliding element 21 thus has a substantially corrugated shape seen in transverse cross-section.

The rib 22 and the concave side faces 23 also have, seen in a sagittal plane (see FIG. 3), a concave curve substantially corresponding to the curve of the femoral implant 2 in that same sagittal plane.

In reference to FIGS. 7 to 11, it appears that the trochlea 12 has, between its anterior end (at the apex of the wall 10) and its posterior end (toward the rear of the branches 11), an evolving shape.

In its upper portion, i.e., its portion cooperating with the sliding element 21 for flexion of the joint from approximately 0° to approximately 50°, the trochlea 12 has a depth smaller than the height of the apex of the rib 22 with respect to the most hollow parts comprised by the side faces 23, and has a width greater than that of said rib 22; the assembly is configured such that the sliding element 21 comes into contact with the femoral implant 2 essentially by that rib 22 and the concave side faces 23 are distant from the condylar surfaces 11a (see FIG. 7). The patellar implant 3 thus has, in the upper portion of the trochlea 12, a possibility of transverse translation with respect to the trochlea 12 in the transverse plane (see FIGS. 7A and 7B), and the possibility of tilting with respect to that trochlea 12, also in the transverse plane (see FIGS. 7C and 7D).

The depth of the trochlea 12 increases gradually toward the lower portion of the trochlea (see FIG. 8), until it corresponds to the height of the rib 22 around 50° of flexion, and the width of that trochlea decreases gradually toward the lower portion of the trochlea, until it corresponds to the width of the rib 22 at that same area around 50° of flexion.

In its lower portion, i.e., its portion cooperating with the sliding element 23 for flexion of the joint going from approximately 50° to approximately 120° (see FIGS. 9 to 11), the trochlea 12 has a depth that becomes greater than the height of the rib 22 with respect to the concave side faces 23 and a width substantially equal to that of that same rib 22. The sliding element 21 therefore comes into contact with the femoral implant 2 essentially by the concave side faces 23, at the condylar faces 11a, and the rib 22, between 50° and 60 to 65°, is separated from the bottom of the trochlea 12 (see FIG. 9).

Thus, in the prosthesis 1, a gradual transfer occurs from the surface of the sliding element 21 coming into contact with the femoral implant 2, said transfer occurring from the central rib 22, which is essentially active at the upper portion of the trochlea 12, toward the concave side faces 23, which are active at the lower portion of that trochlea. The aforementioned possibility of transverse translation and tilting, in addition to the possibility of pivoting of the sliding element 21 with respect to the base 20, is fully favorable to an optimal cooperation of the patellar implant 3 with the femoral implant 2, and therefore an optimal operation of the prosthetic joint.

Furthermore, the rib 22 is, between approximately 50° and 65° of flexion, separated from the bottom of the trochlea 12 and therefore does not risk undergoing a grating effect that would otherwise risk occurring in the lower edge 10a, that lower edge being able to be relatively aggressive and a source of wear on the rib.

Regarding the other parts of said set of elements, the tibial implant 4, which is also made from a metal material, comprises a hollow lower stud 24, for insertion into the medullary channel of a tibia and an upper plate 25 designed to receive the intermediate sliding plate 5. In the primary or initial prosthesis 1, the lower stud 24 receives said rounded end tip 26, fitted into a distal conical cavity comprised by said stud.

The intermediate sliding plate 5 is made from a material favoring sliding, in particular high-density polyethylene. Aside from the stud 15, it forms two articular side surfaces 5a, cooperating with the condylar surfaces 11a, and comprises a lower rod 27 designed to be received in an adjusted manner in the well inwardly formed by the lower stud 24 of the tibial implant 4, to allow the guided pivoting of the plate 5 with respect to the implant 4.

The intercondylar cage 6 is designed to be removably mounted on the femoral implant 2, using two side tabs that it comprises and two screws (not shown), or alternatively, two anchoring studs 7. Said side tabs are designed to be received in corresponding cavities in the distal face 2a of the femoral implant 2, and the anchoring studs 7 comprise threaded lower portions capable of being engaged through holes formed in said side tabs, then in tapped holes emerging in the bottom of said cavities.

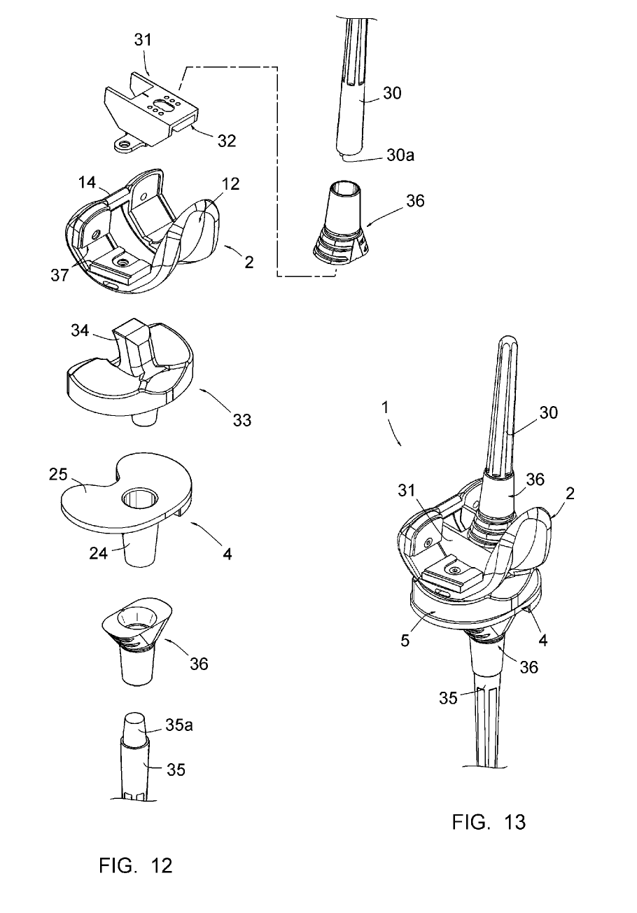

The intercondylar cage 6 is sized so as to be able to house the upper part of the posterior stabilization stud 15. Its anterior end has an interlocking recess 28 capable of allowing the interlocking of said anterior end on the prominence 13. This interlocking makes it possible to position the intercondylar cage 6 on the femoral implant 2 quickly and easily, the latter being completely immobilized with respect to the femoral implant 2 and being able to be used as a solid base for assembling a medullary anchoring rod 30 to the femur, as shown in FIGS. 12 and 13.

As shown in FIG. 1, the upper wall of the cage 6 comprises a series of three secant holes aligned in the anteroposterior direction, bordered by two lateral series of three small holes; the medullary anchoring rod 30 has an inclined face at its end designed to be connected to the cage 6, and comprises a central tapped hole emerging in said face and two small side studs 30a protruding from that face, diametrically opposite; said central tapped hole is designed to receive the screw engaged in one or the other of the three secant holes comprised by the cage 6 so as to ensure fastening of the rod 30 and one of the three positions thus made possible in the anteroposterior direction, and said small side studs 30a are designed to be engaged in two of the holes of said lateral series of small holes, so as to allow reversible positioning of the rod 30 and therefore use of a same rod for a right or left prosthesis.

In reference to FIGS. 12 and 13, it appears that the revision prosthesis 1 may be obtained from the same femoral implant 2 and the same tibial implant 4 as previously described. In that case, the following other elements are used that are comprised by the set of elements according to the invention:

a second intercondylar cage 31, with a height greater than that of the intercondylar cage 6, and whereof the anterior end has an interlocking recess 32 capable of allowing the interlocking of said anterior end on the interlocking prominence 13; said second intercondylar cage 31 comprises the same series of three secant holes aligned in the anteroposterior direction, and the same lateral series of three small holes as the cage 6;

a second intermediate plate 33 comprising a posterior stabilization stud 34 having a height greater than that of the stud 15;

a tibial medullary rod 35, comprising a conical tip 35a capable of being narrowly inserted into the distal conical cavity of the stud 24;

two sleeves 36 with flared or conical base portions, capable of being engaged on the respective medullary rods 30, 35;

lifting blocks 37, which can be fixed on one or more of the interfaces of the femoral element 2 so as to overcome any bone deficiency.

As appears from the preceding, the invention provides a total prosthesis of the knee including a patellar implant 3 in which an optimal cooperation of the patellar implant with the femoral implant 2 is obtained, and in which the sliding element 21 comprised by said patellar implant 3 suffers little or no wear.

The invention also provides a set of modular elements making it possible, simply and quickly, from the same basic elements, to obtain a non-posterior stabilized prosthesis 1, i.e., implanted with retention of the posterior cruciate ligament, a posterior stabilized prosthesis 1 for first-line use or a posterior stabilized revision prosthesis 1.

The invention has been described above in reference to embodiments provided as examples. It is of course not limited to these embodiments, but on the contrary encompasses any other embodiments covered by the appended claims.

What is claimed is:

1. A total knee prosthesis, including a femoral implant which comprises an anterior wall that forms the upper portion of a prosthetic trochlea, and two curved branches extending from the lower edge of said anterior wall, delimiting the lower portion of the prosthetic trochlea between them and forming condylar surfaces between them;
wherein
the trochlea has, in a transverse cross-section, a middle furrow bordered by two rounded convex surfaces,
the trochlea has between an anterior end and a posterior end, the following evolving shape:
in an upper portion, portion cooperating with the patella for flexion of the joint going from approximately 0° to approximately 50°, the trochlea has a depth smaller than the height of the longitudinal middle rib comprised by the patella with respect to the concave side faces bordering that rib and has a width larger than that of that same rib, such that the patella comes into contact with the femoral implant essentially by that rib and said concave side faces are situated at a distance from said rounded convex surfaces; said depth increases gradually toward the lower portion of the trochlea, until it corresponds to the height of said rib at the junction of said upper portion of the trochlea with said lower portion of that trochlea, and said width decreases gradually toward said lower portion of the trochlea, until it corresponds, at that same junction, to the width of said rib;
in a lower portion, the portion of the trochlea cooperating with the patella for flexion of the joint going from approximately 50° to approximately 120°, the trochlea has a depth greater than the height of said rib with respect to said concave side faces and has a width substantially equal to that of said same rib, such that the patella comes into contact with the femoral implant essentially by said concave side faces, said round convex surfaces, and said rib is at a distance from the bottom of the trochlea.

2. The total knee prosthesis according to claim 1, wherein the prosthesis comprises a patellar implant including a base designed to be fastened to the bony patella, duly resected, and a sliding element designed to be mounted on the base, with the possibility of pivoting with respect thereto; the sliding element forms, on the face thereof designed to come into contact with the femoral implant, a longitudinal rib and two concave side faces situated on either side of that rib, reproducing the rib and homologous faces of a bony patella, the rib having a rounded shape in the transverse plane, and its flanks being arched so as to connect continuously with said concave side faces; said rib and said concave side faces also have, in a sagittal plane, a concave curve substantially corresponding to the curve of the trochlea in that same sagittal plane.

3. The total knee prosthesis according to claim 1, wherein said anterior wall of the femoral implant forms an extended bottom of the trochlea, extends in the direction of the lower portion of the trochlea such that the lower edge of that interior wall is at the portion of the trochlea cooperating with the patella for flexion of the joint of approximately 60° to 65°.

4. The total knee prosthesis according to claim 2, wherein the patellar implant comprises means for limiting the pivoting of the sliding element with respect to said base, preventing the pivoting of that sliding element beyond approximately 20° on either side of the neutral position in which said rib is parallel to the trochlea.

5. The total knee prosthesis according to claim 4, wherein said base comprises a slug protruding from its face receiving said sliding element, and said sliding element comprises a lumen in the shape of an arc circle centered on the pivot axis, capable of receiving the slug when the sliding element is mounted on said base.

6. A set of modular elements making it possible to obtain the total knee prosthesis according to claim 3, wherein the set comprises:
a femoral implant including a posterior intercondylar bar and having said extended trochlea bottom, said anterior wall of said femoral implant having, on the underside of its anterior wall, an interlocking prominence;
a first intermediate plate including a posterior stabilization stud; and
a first removable intercondylar cage, capable of housing said posterior stabilization stud, whereof an anterior end has an interlocking recess capable of allowing interlocking of the interior end on said interlocking prominence.

7. The set of modular elements according to claim 6, wherein it comprises a second intercondylar cage, with a height greater than that of said first intercondylar cage, and a second intermediate plate comprising a posterior stabilization stud with a height greater than that of the posterior stabilization stud of said first intermediate plate, the anterior end of said second intercondylar cage having an interlocking recess capable of allowing the interlocking of said anterior end on said interlocking prominence.

8. The set of modular elements according to claim 7, wherein the upper wall of said first and/or said second intercondylar cage comprises means for mounting a rod or a medullary anchoring stud according to a plurality of possible positions in the anteroposterior direction.

9. The set of modular elements according to claim 8, wherein:
the upper wall of said first and/or said second intercondylar cage comprises a series of three secant holes aligned in the anteroposterior direction, bordered by two lateral series of three small holes;
the medullary anchoring rod has an inclined face at its end designed to be connected to the cage, and comprises a central tapped hole emerging in said face and two small side studs protruding from that face, diametrically opposite; said central tapped hole is designed to receive the screw engaged in one or the other of the three secant holes comprised by the cage so as to ensure fastening of the rod and one of the three positions thus made possible in the anteroposterior direction, and said small side studs are designed to be engaged in two of the holes of said lateral series of small holes, so as to allow reversible positioning of the rod and therefore use of a same rod for a right or left prosthesis.

10. The set of modular elements according to claim 6, wherein it comprises at least one medullary anchoring rod and at least one sleeve with a flared or conical base portion, capable of being engaged on said rod.
